(19) United States
(12) Patent Application Publication
(10) Pub. No.: US 2012/0090983 A1
(43) Pub. Date: Apr. 19, 2012

(54) Continuous Method For Acylating Amino Group-Carrying Organic Acids

(75) Inventors: Matthias Krull, Harxheim (DE); Roman Morschhaeuser, Mainz (DE)

(73) Assignee: CLARIANT FINANCE (BVI) LIMITED, Tortola (VG)

(21) Appl. No.: 13/378,181

(22) PCT Filed: Jun. 9, 2010
(86) PCT No.: PCT/EP2010/003444

§ 371 Date: Dec. 14, 2011

(30) Foreign Application Priority Data
Jun. 30, 2009 (DE) 10 2009 031 056.8

(51) Int. Cl. B01J 19/12; C07F 9/02; C07C 309/01; C07C 209/68

(52) U.S. Cl. 204/157.73; 204/157.81; 204/157.77

(57) ABSTRACT

The invention relates to a continuous method for N-acylating amino group-carrying organic acids by reacting at least one carboxylic acid of formula (I) R¹—COOH (I), wherein R¹ represents hydrogen or an optionally substituted hydrocarbon group with 1 to 50 carbon atoms, with at least one at least one amino group-carrying organic acid of formula (II) R²NH-A-X (II), wherein A represents an optionally substituted hydrocarbon group with 1 to 50 carbon atoms, X represents an acid group or the metal salt thereof and R² represents hydrogen, an optionally substituted hydrocarbon group with 1 to 50 C atoms or a group of the formula -A-X, wherein A and X independently are defined as above, in a reaction tube the longitudinal axis of which extends in the direction of propagation of the microwaves of a monomode microwave applicator, under microwave irradiation to form amide.

The present invention relates to a continuous process for acylation of organic acids bearing amino groups under microwave irradiation on the industrial scale.

Acylation products of organic acids bearing amino groups find various uses as chemical raw materials. For instance, organic acids which bear amino groups and have been N-acylated with lower carboxylic acids are of particular interest as pharmaceuticals or as intermediates for the production of pharmaceuticals. Organic acids which bear amino groups and have been N-acylated with relatively long-chain fatty acids have amphiphilic properties, and they therefore find various uses as a constituent in washing and cleaning compositions and in cosmetics. In addition, they are used successfully as an auxiliary in metalworking, in the formulation of crop protection compositions, as antistats for polyolefins, and in the production and processing of mineral oil.

In the industrial preparation of N-acylation products of acids bearing amino groups, a reactive derivative of a carboxylic acid, such as acid anhydride, acid chloride or ester, is typically reacted with the acid bearing at least one amino group, usually working in an alkali medium. This leads firstly to high production costs and secondly to unwanted accompanying products, for example salts or acids, which have to be removed and disposed of or worked up. For example, the Schotten-Baumann synthesis, by which numerous amides of amines bearing acid groups are prepared on the industrial scale, forms at least equimolar amounts of sodium chloride. The use of coupling reagents such as N,N′-dicyclohexylcarbodiimide (DCC), which is likewise practised, is expensive, requires special measures due to the toxicity of the coupling reagents and conversion products thereof, and likewise leads to large amounts of by-products for disposal. The desirable direct thermal condensation of carboxylic acid and amine bearing at least one acid group requires very high temperatures and long reaction times, but only moderate yields are obtained (J. Am. Chem. Soc., 59 (1937), 401-402). Under these reaction conditions, the corrosivity of the reaction mixtures of acid, amine, amide and water of reaction additionally presents great technical problems since these mixtures severely attack or dissolve metallic reaction vessels at the high reaction temperatures required. The metal contents introduced into the products as a result are very undesirable since they impair the product properties not just with regard to the color thereof, but also catalyze decomposition reactions and hence reduce the yield. The latter problem can be circumvented to some degree by using special reaction vessels made of materials with high corrosion resistance, or with appropriate coatings, but this nevertheless requires long reaction times and thus leads to products of impaired color. Furthermore, the separation of carboxylic acid used and amide formed is often exceptionally difficult, since the two frequently have very similar boiling points and additionally form azeotropes.

A more recent approach to the synthesis of amides is the microwave-supported reaction of carboxylic acids and amines to give amides.

Vázquez-Tato, Synlett 1993, 506, discloses the use of microwaves as a heat source for the preparation of amides from carboxylic acids and arylaliphatic amines via the ammonium salts. The syntheses are effected on the mmol scale.

Gelens et al., Tetrahedron Letters 2005, 46(21), 3751-3754, disclose a multitude of amides which have been synthesized with the aid of microwave radiation. The syntheses are effected in 10 ml vessels. Amines bearing acid groups are not used.

The scaleup of such microwave-supported reactions from the laboratory to an industrial scale and hence the development of plants suitable for production of several tonnes, for example several tens, several hundreds or several thousands of tonnes, per year with space-time yields of interest for industrial scale applications has, however, not been achieved to date. One reason for this is the penetration depth of microwaves into the reaction mixture, which is typically limited to several millimeters to a few centimeters, and causes restriction to small vessels especially in reactions performed in batchwise processes, or leads to very long reaction times in stirred reactors. The occurrence of discharge processes and plasma formation places tight limits on an increase in the field strength, which is desirable for the irradiation of large amounts of substance with microwaves, especially in the multimode units used with preference to date for scaleup of chemical reactions. Moreover, scaleup problems are presented by the inhomogeneity of the microwave field, which leads to local overheating of the reaction mixture in multimode microwave systems and is caused by more or less uncontrolled reflections of the microwaves injected into the microwave oven at the walls thereof and the reaction mixture. In addition, the microwave absorption coefficient of the reaction mixture, which often changes during the reaction, presents difficulties with regard to a safe and reproducible reaction regime.

Chen et al. (J. Chem. Soc., Chem. Commun., 1990, 807-809), describe a continuous laboratory microwave reactor in which the reaction mixture is conducted through a Teflon tube coil mounted in a microwave oven. A similar continuous laboratory microwave reactor is described by Cablewski et al. (J. Org. Chem. 1994, 59, 3408-3412) for performance of a wide variety of different chemical reactions. In both cases, the microwave operated in multimode, however, does not allow up-scaling to the industrial scale range for the reasons described above. In addition, the efficiency of these processes with regard to the microwave absorption of the reaction mixture is low due to the more or less homogeneous distribution of microwave energy over the applicator space in multimode microwave applicators, and the lack of focus of the microwave energy on the tube coil. A significant increase in the microwave power injected can lead to unwanted plasma discharges or to what are called thermal runaway effects. In addition, the spatial inhomogeneities of the microwave field in the applicator space, which change with time and are referred to as hotspots, make a reliable and reproducible reaction regime on a large scale impossible.

Additionally known are monomode or single-mode microwave applicators which work with a single microwave mode which propagates in only one spatial direction and is focused onto the reaction vessel by waveguides of exact dimensions. This equipment allows higher local field strengths, but has to date been restricted to small reaction volumes (ml) on the laboratory scale due to the geometric requirements (for example, the intensity of the electrical field is at its greatest at its wave crests and approaches zero at the node points).

A process for preparing N-acylation products of organic acids bearing amino groups was therefore sought, in which carboxylic acid and organic acid bearing amino groups can be converted to the amide under microwave irradiation even on the industrial scale. This should achieve very high, i.e. up to quantitative, conversion levels and yields. The process should additionally enable a very energy-saving preparation of the amides, which means that the microwave power used should be absorbed very substantially quantitatively by the reaction mixture and the process should give a high energy efficiency. At the same time, only minor amounts, if any, of by-products should be obtained. The amides should also have a minimum content of catalytically active metal ions, especially of the transition group metals, for example, iron, and low intrinsic color. In addition, the process should ensure a reliable and reproducible reaction regime.

It has been found that, surprisingly, N-acylation products of organic acids bearing amino groups can be prepared in industrially relevant amounts by direct reaction of carboxylic acids with organic acids bearing amino groups in a continuous process by only briefly heating by means of irradiation with microwaves in a reaction tube whose longitudinal axis is in the direction of propagation of the microwaves of a monomode microwave applicator. At the same time, the microwave energy injected into the microwave applicator is absorbed virtually quantitatively by the reaction mixture. The process according to the invention additionally has a high level of reliability in execution and gives high reproducibility of the reaction conditions established. The N-acylation products of organic acids bearing acid groups prepared by the process according to the invention exhibit a high purity and low intrinsic color not obtainable in comparison to by conventional preparation processes without additional process steps.

The invention provides a continuous process for N-acylation of organic acids bearing amino groups, in which at least one carboxylic acid of the formula (I)

R¹—COOH (I)

in which

R¹ is hydrogen or an optionally substituted hydrocarbyl radical having 1 to 50 carbon atoms,

is reacted with at least one organic acid which bears at least one amino group and is of the formula (II)

R²NH-A-X (II)

in which

A is an optionally substituted hydrocarbyl radical having 1 to 50 carbon atoms,

X is an acid group or the metal salt thereof, and

R² is hydrogen, an optionally substituted hydrocarbyl radical having 1 to 50 carbon atoms or a group of the formula -A-X in which A and also X are each independently as defined above,

under microwave irradiation in a reaction tube whose longitudinal axis is in the direction of propagation of the microwaves from a monomode microwave applicator to give the amide.

Suitable carboxylic acids of the formula I are generally compounds which have at least one carboxyl group on an optionally substituted hydrocarbyl radical having 1 to 50 carbon atoms, and formic acid. The hydrocarbyl radical may be aliphatic or aromatic.

In a first preferred embodiment, the hydrocarbyl radical R¹ is an aliphatic unsubstituted alkyl or alkenyl radical. In a further preferred embodiment, the aliphatic hydrocarbyl radical bears one or more, for example two, three, four or more, further substituents. Suitable substituents are, for example, halogen atoms, halogenated alkyl radicals, C₁-C₅-alkoxy, for example methoxy, poly(C₁-C₅-alkoxy), poly(C₁-C₅-alkoxy)alkyl, carboxyl, amide, cyano, nitrile, nitro and/or aryl groups having 5 to 20 carbon atoms, for example phenyl groups, with the proviso that these substituents are stable under reaction conditions and do not enter into any side reactions, for example elimination reactions. The C₅-C₂₀-aryl groups may themselves in turn bear substituents, for example halogen atoms, halogenated alkyl radicals, C₁-C₂₀-alkyl, C₂-C₂₀-alkenyl, C₁-C₅-alkoxy, for example methoxy, ester, amide, cyano, nitrile and/or nitro groups. However, the aliphatic hydrocarbyl radical bears at most as many substituents as it has valences. In a specific embodiment, the aliphatic hydrocarbyl radical R¹ has one or more further carboxyl groups. Thus, the process according to the invention is likewise suitable for N-acylation of organic acids bearing amino groups with polycarboxylic acids which bear, for example, two, three, four or more carboxyl groups. The carboxyl groups of the polycarboxylic acid (I) can be completely or else only partly amidated. The amidation level can be adjusted, for example, through the stoichiometry between carboxylic acid (I) and of organic acid (II) bearing amino groups in the reaction mixture. Additionally preferably, the aliphatic hydrocarbyl radical R¹ does not bear any amino groups.

Particular preference is given in accordance with the invention to carboxylic acids (I) which bear an aliphatic hydrocarbyl radical having 1 to 30 carbon atoms and especially having 2 to 24 carbon atoms, for example having 3 to 20 carbon atoms. They may be of natural or synthetic origin. The aliphatic hydrocarbyl radical may also bear heteroatoms, for example oxygen, nitrogen, phosphorus and/or sulfur, but preferably not more than one heteroatom per 3 carbon atoms. The aliphatic hydrocarbyl radicals may be linear, branched or cyclic. The carboxyl group may be bonded to a primary, secondary or tertiary carbon atom. It is preferably bonded to a primary carbon atom. The hydrocarbyl radicals may be saturated or, if their hydrocarbyl radical R¹ comprises at least 2 carbon atoms, also unsaturated. Unsaturated hydrocarbyl radicals preferably contain one or more C═C double bonds and more preferably one, two or three C═C double bonds. Thus, the process according to the invention has been found to be particularly useful for preparation of amides of polyunsaturated carboxylic acids, since the double bonds of the unsaturated carboxylic acids are not attacked under the reaction conditions of the process according to the invention. Preferred cyclic aliphatic hydrocarbyl radicals have at least one ring having four, five, six, seven, eight or more ring atoms.

In a preferred embodiment, R¹ is a saturated alkyl radical having 1, 2, 3 or 4 carbon atoms. This may be linear or else branched. The carboxyl group may be bonded to a primary, secondary or, as in the case of pivalic acid, tertiary carbon atom. In a particularly preferred embodiment, the alkyl radical is an unsubstituted alkyl radical. In a further particularly preferred embodiment, the alkyl radical bears one to nine, preferably one to five, for example two, three or four, further substituents. Preferred further substituents are carboxyl groups and optionally substituted C₅-C₂₀-aryl radicals.

In a further preferred embodiment, the carboxylic acid (I) is an ethylenically unsaturated carboxylic acid. In this case, R¹ is an optionally substituted alkenyl group having 2 to 4 carbon atoms. Ethylenically unsaturated carboxylic acids are understood here to mean those carboxylic acids which have a C═C double bond conjugated to the carboxyl group. The alkenyl group may be linear or, if it comprises at least three carbon atoms, branched. In a preferred embodiment, the alkenyl radical is an unsubstituted alkenyl radical. More preferably, R¹ is an alkenyl radical having 2 or 3 carbon atoms. In a further preferred embodiment, the alkenyl radical bears one or more, for example two, three or more, further substituents. However, the alkenyl radical bears at most as many substituents as it has valences. In particularly preferred embodiments, the alkenyl radical R¹ bears, as further substituents, a carboxyl group or an optionally substituted C₅-C₂₀-aryl group. Thus, the process according to the invention is equally suitable for conversion of ethylenically unsaturated dicarboxylic acids.

In a further preferred embodiment, the carboxylic acid (I) is a fatty acid. In this case, R¹ is an optionally substituted aliphatic hydrocarbyl radical having 5 to 50 carbon atoms. Particular preference is given to fatty acids which bear an aliphatic hydrocarbyl radical having 6 to 30 carbon atoms and especially having 7 to 26 carbon atoms, for example having 8 to 22 carbon atoms. In a preferred embodiment, the hydrocarbyl radical of the fatty acid is an unsubstituted alkyl or alkenyl radical. In a further preferred embodiment, the hydrocarbyl radical of the fatty acid bears one or more, for example two, three, four or more, further substituents. In a specific embodiment, the hydrocarbyl radical of the fatty acid bears one, two, three, four or more further carboxyl groups.

In a further preferred embodiment, the hydrocarbyl radical R¹ is an aromatic radical. Aromatic carboxylic acids (I) are understood here generally to mean compounds which bear at least one carboxyl group bonded to an aromatic system. Aromatic systems are understood to mean cyclic, through-conjugated systems having (4n+2) π electrons where n is a natural integer and is preferably 1, 2, 3, 4 or 5. The aromatic system may be mono- or polycyclic, for example di- or tricyclic. The aromatic system is preferably formed from carbon atoms. In a further preferred embodiment, as well as carbon atoms, it contains one or more heteroatoms, for example nitrogen, oxygen and/or sulfur. Examples of such aromatic systems are benzene, naphthalene, phenanthrene, indole, furan, pyridine, pyrrole, thiophene and thiazole. The aromatic system may, as well as the carboxyl group, bear one or more, for example one, two, three or more, identical or different further substituents. Suitable further substituents are, for example, halogen atoms, alkyl and alkenyl radicals, and also hydroxyl, hydroxyalkyl, alkoxy, poly(alkoxy), amide, cyano and/or nitrile groups. These substituents may be bonded to any position on the aromatic system. However, the aryl radical bears at most as many substituents as it has valences. Preferably, the aryl radical does not bear any amino groups.

In a specific embodiment, the aryl radical of the aromatic carboxylic acid (I) bears further carboxyl groups. Thus, the process according to the invention is likewise suitable for conversion of aromatic carboxylic acids having, for example, two or more carboxylic acid groups. In the process according to the invention, the carboxylic acid groups can be converted completely or else only partially to amides. The degree of amidation can be adjusted, for example, through the stoichiometry between carboxylic acid and organic acid bearing amino groups in the reaction mixture.

In addition, the process according to the invention is particularly suitable for preparation of alkylarylcarboxamides, for example alkylphenylcarboxamides. In the process according to the invention, aromatic carboxylic acids (I) in which the aryl radical bearing the carboxylic acid group additionally bears at least one alkyl or alkylene radical are reacted with organic acids (II) bearing amino groups. The process is particularly advantageous for preparation of alkylbenzamides whose aryl radical bears at least one alkyl radical having 1 to 20 carbon atoms and especially 1 to 12 carbon atoms, for example 1 to 4 carbon atoms.

The process according to the invention is additionally particularly suitable for preparation of aromatic carboxamides whose aryl radical R¹ bears one or more, for example two or three, hydroxyl groups and/or hydroxyalkyl groups. In the reaction of the corresponding carboxylic acids (I), especially with at most equimolar amounts of organic acids bearing amino groups of the formula (II), selective amidation of the carboxyl group and no aminolysis of the phenolic OH group takes place.

Examples of carboxylic acids (I) suitable for amidation by the process according to the invention include formic acid, acetic acid, propionic acid, butyric acid, isobutyric acid, pentanoic acid, isopentanoic acid, pivalic acid, acrylic acid, methacrylic acid, crotonic acid, 2,2-dimethylacrylic acid, maleic acid, fumaric acid, itaconic acid, cinnamic acid, methoxycinnamic acid, succinic acid, butanetetracarboxylic acid, phenylacetic acid, (2-bromophenyl)acetic acid, (methoxyphenyl)acetic acid, (dimethoxyphenyl)acetic acid, 2-phenyl propionic acid, 3-phenylpropionic acid, 3-(4-hydroxyphenyl)propionic acid, 4-hydroxyphenoxyacetic acid, hexanoic acid, cyclohexanoic acid, heptanoic acid, octanoic acid, nonanoic acid, neononanoic acid, decanoic acid, neodecanoic acid, undecanoic acid, neoundecanoic acid, dodecanoic acid, tridecanoic acid, isotridecanoic acid, tetradecanoic acid, 12-methyltridecanoic acid, pentadecanoic acid, 13-methyltetradecanoic acid, 12-methyltetradecanoic acid, hexadecanoic acid, 14-methylpentadecanoic acid, heptadecanoic acid, 15-methylhexadecanoic acid, 14-methylhexadecanoic acid, octadecanoic acid, isooctadecanoic acid, eicosanoic acid, docosanoic acid, tetracosanoic acid, myristoleic acid, palmitoleic acid, hexadecadienoic acid, delta-9-cis-heptadecenoic acid, oleic acid, petroselic acid, vaccenic acid, linoleic acid, linolenic acid, gadoleic acid, gondoic acid, eicosadienoic acid, arachidonic acid, cetoleic acid, erucic acid, docosadienic acid, tetracosenoic acid, dodecenylsuccenic acid and octadecenylsuccenic acid and dimer fatty acids preparable from unsaturated fatty acids and mixtures thereof. Additionally suitable are carboxylic acid mixtures obtained from natural fats and oils, for example cottonseed oil, coconut oil, peanut oil, safflower oil, corn oil, palm kernel oil, rapeseed oil, olive oil, mustardseed oil, soybean oil, sunflower oil, and also tallow oil, bone oil and fish oil. Likewise suitable as carboxylic acids or carboxylic acid mixtures for the process according to the invention are tall oil fatty acid, and resin acids and naphthenic acids. Examples of further carboxylic acids (I) suitable for amidation by the process according to the invention include benzoic acid, phthalic acid, isophthalic acid, the different isomers of naphthalenecarboxyiic acid, pyridinecarboxylic acid and naphthalenedicarboxylic acid, and from trimellitic acid, trimesic acid, pyromellitic acid and mellitic acid, the different isomers of methoxybenzoic acid, hydroxybenzoic acid, hydroxymethylbenzoic acid, hydroxymethoxybenzoic acid, hydroxydimethoxybenzoic acid, hydroxyisophthalic acid, hydroxynaphthalenecarboxylic acid, hydroxypyridinecarboxylic acid and hydroxymethylpyridinecarboxylic acid, hydroxyquinolinecarboxylic acid, and from o-toluic acid, m-toluic acid, p-toluic acid, o-ethylbenzoic acid, m-ethylbenzoic acid, p-ethylbenzoic acid, o-propylbenzoic acid, m-propylbenzoic acid, p-propylbenzoic acid and 3,4-dimethylbenzoic acid. Mixtures of different aryl and/or alkylarylcarboxylic acids are equally suitable.

The organic acid (II) bearing at least one amino group bears at least one acidic X group bonded to the nitrogen of the amino group via the optionally substituted hydrocarbyl radical A. Acidic X groups are understood to mean functional groups which can eliminate at least one acidic proton. Acidic X groups preferred in accordance with the invention are carboxylic acids and organic acids of sulfur and phosphorus, for example sulfonic acids and phosphonic acids.

The hydrocarbyl radical A is preferably an aliphatic or aromatic radical, with the proviso that A is not an acyl group or a hydrocarbyl radical bonded to the nitrogen via an acyl group.

In a first preferred embodiment, A is an aliphatic radical having 1 to 12 and more preferably having 2 to 6 carbon atoms. It may be linear, cyclic and/or branched. It is preferably saturated. A may bear further substituents. Suitable further substituents are, for example, carboxamides, guanidine radicals, optionally substituted C₆-C₁₂-aryl radicals, for example indole and imidazole, and acid groups, for example carboxylic acids and/or phosphonic acid groups. The A radical may also bear hydroxyl groups, in which case, however, the reaction has to be effected with at most equimolar amounts of carboxylic acids (I) in order to avoid acylation of these OH groups. In a particularly preferred embodiment, the aliphatic A radical bears the acid group X on the α- or β-carbon atom to the nitrogen atom. The process according to the invention has been found to be particularly useful for acylation of aliphatic acids bearing amino groups, in which A is an alkyl radical having 1 to 12 carbon atoms and in which the acid group X is on the α- or 62 -carbon atoms to the nitrogen atom, and especially of α-aminocarboxylic acids, β-aminosulfonic acids and aminomethylenephosphonic acids.

In a further preferred embodiment, A is an aromatic hydrocarbyl radical having 5 to 12 carbon atoms. Aromatic systems are understood here to mean cyclic, through-conjugated systems having (4n+2) π electrons in which n is a natural integer and is preferably 1, 2, 3, 4 or 5. The aromatic system may be mono- or polycyclic, for example di- or tricyclic; it is preferably monocyclic. The aromatic A radical may contain one or more heteroatoms, for example oxygen, nitrogen and/or sulfur. The amino and acid groups of this aromatic acid (II) bearing at least one amino group may be arranged in ortho, meta or para positions on the aromatic system and, in the case of polycyclic aromatic systems, may also be present on different rings. Examples of suitable aromatic systems A are benzene, naphthalene, phenanthrene, indole, furan, pyridine, pyrrole, thiophene and thiazole. In addition, the aromatic system A may bear, in addition to carboxyl and amino groups, one or more, for example one, two, three or more, identical or different further substituents. Suitable further substituents are, for example, halogen atoms, alkyl and alkenyl radicals, and hydroxyalkyl, alkoxy, poly(alkoxy), amide, cyano and/or nitrile groups. These substituents may be bonded to any position in the aromatic system. However, the aryl radical bears at most as many substituents as it has valences.

In a preferred embodiment, R² is an aliphatic radical. This has preferably 1 to 24, more preferably 2 to 18 and especially 3 to 6 carbon atoms. The aliphatic radical may be linear, branched or cyclic. It may additionally be saturated or unsaturated; it is preferably saturated. The aliphatic radical may bear substituents, for example halogen atoms, halogenated alkyl radicals, hydroxyl, C₁-C₅-alkoxyalkyl, cyano, nitrile, nitro and/or C₅-C₂₀-aryl groups, for example phenyl radicals. The C₅-C₂₀-aryl radicals may in turn optionally be substituted by halogen atoms, halogenated alkyl radicals, hydroxyl, C₁-C₂₀-alkyl, C₂-C₂₀-alkenyl, C₁-C₅-alkoxy groups, for example methoxy, ester, amide, cyano and/or nitrile groups. Particularly preferred aliphatic R² radicals are methyl, ethyl, n-propyl, isopropyl, n-butyl, isobutyl and tert-butyl, n-hexyl, cyclohexyl, n-octyl, n-decyl, n-dodecyl, tridecyl, isotridecyl, tetradecyl, hexadecyl, octadecyl and methylphenyl, and especially preferred are methyl, ethyl, propyl, and butyl.

In a further preferred embodiment, R² is an optionally substituted C₆-C₁₂-aryl group or an optionally substituted heteroaromatic group having 5 to 12 ring members. Preferred heteroatoms are oxygen, nitrogen and sulfur. In a specific embodiment, R² is a further group of the formula -A-X where both A and X are independently as defined above.

When the hydrocarbyl radicals A and/or R² bear further acid groups, for example carboxyl and/or phosphonic acid groups, measures should be taken to counteract the at least partial occurrence of polycondensation of the organic acid (II) bearing at least one amino group.

In a particularly preferred embodiment, R² is hydrogen.

Examples of organic acids (II) which bear at least one amino group and are suitable in accordance with the invention are amino acids such as glycine, alanine, arginine, asparagine, glutamine, histidine, leucine, isoleucine, valine, phenylalanine, serine, tyrosine, 3-aminopropionic acid (β-alanine), 3-aminobutyric acid, 2-aminobenzoic acid, 4-aminobenzoic acid, 2-aminoethanesulfonic acid (taurine), N-methyltaurine, 2-(aminomethyl)phosphonic acid, 1-aminoethylphosphonic acid, (1-amino-2-methylpropyl)phosphonic acid, (1-amino-1-phosphonooctyl)phosphonic acid.

In the process according to the invention, it is possible to react carboxylic acid (I) and organic acid (II) bearing an amino group with one another in any desired ratios. Preference is given to effecting the reaction between carboxylic acid (I) and organic acid (II) bearing an amino group with molar ratios of 100:1 to 1:10, preferably of 10:1 to 1:2, especially of 3:1 to 1:1.2, based in each case on the molar equivalents of carboxyl groups in (I) and amino groups in (II). in a specific embodiment, carboxylic acid (I) and organic acid (II) bearing an amino group are used in equimolar amounts, based on the molar equivalents of carboxyl groups in (I) and amino groups in (II).

In many cases, it has been found to be advantageous to work with an excess of carboxylic acid (I), i.e. molar ratios of carboxyl groups to amino groups of at least 1.01:1.00 and especially between 50:1 and 1.02:1, for example between 10:1 and 1.1:1. The amino groups are converted virtually quantitatively to the amide. This process is particularly advantageous when the carboxylic acid used is volatile. “Volatile” means here that the carboxylic acid (I) has a boiling point at standard pressure of preferably below 200° C., for example below 160° C., and can thus be removed from the amide by distillation.

The inventive preparation of the amides is effected by reaction of carboxylic acid (I) and organic acid (II) bearing an amino group to give the ammonium salt and subsequent irradiation of the salt with microwaves in a reaction tube whose longitudinal axis is in the direction of propagation of the microwaves in a monomode microwave applicator. In the simplest case, the conversion to the ammonium salt proceeds by mixing carboxylic acid (I) and organic acid (II) bearing an amino group, optionally in the presence of a solvent.

In many cases, it has likewise been found to be useful to convert the organic acid (II) bearing at least one amino group to a metal salt before the reaction, or to use it in the form of a metal salt for reaction with the carboxylic acid (I). Equally, the mixture of (I) and (II) can be admixed with an essentially equimolar amount of base based on the concentration of the acid groups X. Bases preferred for this purpose are especially inorganic bases, for example metal hydroxides, oxides, carbonates, silicates or alkoxides. Particular preference is given to the hydroxides, oxides, carbonates, silicates or alkoxides of alkali metals or alkaline earth metals, for example lithium hydroxide, sodium hydroxide, potassium hydroxide, sodium methoxide, potassium methoxide, sodium tert-butoxide, potassium tert-butoxide, sodium carbonate and potassium carbonate. In a preferred embodiment, the conversion to the ammonium salt is effected by adding a solution of the appropriate base, for example in a lower alcohol, for example methanol, ethanol or propanol or else in water, to one of the reactants or to the reaction mixture. This mode of operation has been found to be useful especially in the case of acylation of amines (II) bearing strong acid groups X, for example amines (II) bearing sulfonic or phosphonic acid groups. Strong acids are understood here to mean especially acids having a pKa of below 3.5 and especially below 3.0.

In a preferred embodiment, the reaction is accelerated or completed by working in the presence of at least one catalyst. Preference is given to working in the presence of a basic catalyst or mixtures of two or more of these catalysts. The basic catalysts used in the context of the present invention are quite generally those basic compounds which are suitable for accelerating the amidation of carboxylic acids with amines to give carboxamides. These substances can be used in solid form, for example as a dispersion or fixed bed, or as a solution, for example as an aqueous or preferably alcoholic solution. Examples of suitable catalysts are inorganic and organic bases, for example metal hydroxides, oxides, carbonates, silicates or alkoxides. In a preferred embodiment, the basic catalyst is selected from the group of the hydroxides, oxides, carbonates, silicates and alkoxides of alkali metals and alkaline earth metals. Very particular preference is given to lithium hydroxide, sodium hydroxide, potassium hydroxide, sodium methoxide, potassium methoxide, sodium tert-butoxide, potassium tert-butoxide, sodium carbonate and potassium carbonate. Cyanide ions are also suitable as a catalyst. Further suitable catalysts are strongly basic ion exchangers. The amount of the catalysts used depends on the activity and stability of the catalyst under the selected reaction conditions and should be matched to the particular reaction. The amount of the catalyst to be used can vary within wide limits. It has often been found to be useful to work with 0.1 to 2.0 mol of base, for example with 0.2 to 1.0 mol of base, per mole of amine used. Particular preference is given to using catalytic amounts of the abovementioned reaction-accelerating compounds, preferably in the range between 0.001 and 10% by weight, more preferably in the range from 0.01 to 5% by weight, for example between 0.02 and 2% by weight, based on the amount of carboxylic acid (I) and acid (II) bearing an amino group used.

The reaction mixture is preferably irradiated with microwaves in a substantially microwave-transparent reaction tube within a hollow conductor connected to a microwave generator. The reaction tube is preferably aligned axially with the central axis of symmetry of the hollow conductor.

The hollow conductor which functions as the microwave applicator is preferably configured as a cavity resonator. Additionally preferably, the microwaves unabsorbed in the hollow conductor are reflected at the end thereof. The length of the cavity resonator is preferably such that a standing wave forms therein. Configuration of the microwave applicator as a resonator of the reflection type achieves a local increase in the electrical field strength at the same power supplied by the generator and increased energy exploitation.

The cavity resonator is preferably operated in E₀₁ₙ mode where n is an integer and specifies the number of field maxima of the microwave along the central axis of symmetry of the resonator. In this operation, the electrical field is directed in the direction of the central axis of symmetry of the cavity resonator. It has a maximum in the region of the central axis of symmetry and decreases to the value 0 toward the outer surface. This field configuration is rotationally symmetric about the central axis of symmetry. Use of a cavity resonator with a length where n is an integer enables the formation of a standing wave. According to the desired flow rate of the reaction mixture through the reaction tube, the temperature required and the residence time required in the resonator, the length of the resonator is selected relative to the wavelength of the microwave radiation used. n is preferably an integer from 1 to 200, more preferably from 2 to 100, particularly from 3 to 50, especially from 4 to 20, for example three, four, five, six, seven, eight, nine or ten.

The E₀₁ₙ mode of the cavity resonator is also referred to in English as the TM₀₁ₙ mode; see, for example, K. Lange, K. H. Löcherer, “Taschenbuch der Hochfrequenztechnik” [Handbook of High-Frequency Technology], volume 2, pages K21 ff.

The microwave energy can be injected into the hollow conductor which functions as the microwave applicator through holes or slots of suitable dimensions. In an embodiment particularly preferred in accordance with the invention, the reaction mixture is irradiated with microwaves in a reaction tube present in a hollow conductor with a coaxial transition of the microwaves. Microwave devices particularly preferred for this process are formed from a cavity resonator, a coupling device for injecting a microwave field into the cavity resonator and with one orifice each on two opposite end walls for passage of the reaction tube through the resonator. The microwaves are preferably injected into the cavity resonator by means of a coupling pin which projects into the cavity resonator. The coupling pin is preferably configured as a preferably metallic inner conductor tube which functions as a coupling antenna. In a particularly preferred embodiment, this coupling pin projects through one of the end orifices into the cavity resonator. The reaction tube more preferably adjoins the inner conductor tube of the coaxial transition, and is especially conducted through the cavity thereof into the cavity resonator. The reaction tube is preferably aligned axially with a central axis of symmetry of the cavity resonator. For this purpose, the cavity resonator preferably has one central orifice each on two opposite end walls for passage of the reaction tube.

The microwaves can be fed into the coupling pin or into the inner conductor tube which functions as a coupling antenna, for example, by means of a coaxial connecting line. In a preferred embodiment, the microwave field is supplied to the resonator via a hollow conductor, in which case the end of the coupling pin projecting out of the cavity resonator is conducted into the hollow conductor through an orifice in the wall of the hollow conductor, and takes microwave energy from the hollow conductor and injects it into the resonator.

In a specific embodiment, the reaction mixture is irradiated with microwaves in a microwave-transparent reaction tube which is axially symmetric within an E₀₁ₙ round hollow conductor with a coaxial transition of the microwaves. In this case, the reaction tube is conducted through the cavity of an inner conductor tube which functions as a coupling antenna into the cavity resonator. In a further preferred embodiment, the reaction mixture is irradiated with microwaves in a microwave-transparent reaction tube which is conducted through an E₀₁ₙ cavity resonator with axial introduction of the microwaves, the length of the cavity resonator being such as to form n=2 or more field maxima of the microwave. In a further preferred embodiment, the reaction mixture is irradiated with microwaves in a microwave-transparent reaction tube which is conducted through an E₀₁ₙ cavity resonator with axial introduction of the microwaves, the length of the cavity resonator being such as to form a standing wave where n=2 or more field maxima of the microwave. In a further preferred embodiment, the reaction mixture is irradiated with microwaves in a microwave-transparent reaction tube which is axially symmetric within a circular cylindrical E₀₁ₙ cavity resonator with a coaxial transition of the microwaves, the length of the cavity resonator being such as to form n=2 or more field maxima of the microwave. In a further preferred embodiment, the reaction mixture is irradiated with microwaves in a microwave-transparent reaction tube which is axially symmetric within a circular cylindrical E₀₁ₙ cavity resonator with a coaxial transition of the microwaves, the length of the cavity resonator being such as to form a standing wave where n=2 or more field maxima of the microwave.

Microwave generators, for example the magnetron, the klystron and the gyrotron, are known to those skilled in the art.

The reaction tubes used to perform the process according to the invention are preferably manufactured from substantially microwave-transparent, high-melting material. Particular preference is given to using nonmetallic reaction tubes. “Substantially microwave-transparent” is understood here to mean materials which absorb a minimum amount of microwave energy and convert it to heat. A measure employed for the ability of a substance to absorb microwave energy and convert it to heat is often the dielectric loss factor tan δ=ε″/ε′. The dielectric loss factor tan δ is defined as the ratio of dielectric loss ε″ to dielectric constant ε′. Examples of tan δ values of different materials are reproduced, for example, in D. Bogdal,

Microwave-assisted Organic Synthesis, Elsevier 2005. For reaction tubes suitable in accordance with the invention, materials with tan δ values measured at 2.45 GHz and 25° C. of less than 0.01, particularly less than 0.005 and especially less than 0.001 are preferred. Preferred microwave-transparent and thermally stable materials include primarily mineral-based materials, for example quartz, aluminum oxide, sapphire, zirconium oxide, silicon nitride and the like. Other suitable tube materials are thermally stable plastics, such as especially fluoropolymers, for example Teflon, and industrial plastics such as polypropylene, or polyaryl ether ketones, for example glass fiber-reinforced polyetheretherketone (PEEK). In order to withstand the temperature conditions during the reaction, minerals, such as quartz or aluminum oxide, coated with these plastics have been found to be especially suitable as reactor materials.

Reaction tubes particularly suitable for the process according to the invention have an internal diameter of one millimeter to approx. 50 cm, particularly between 2 mm and 35 cm, especially between 5 mm and 15 cm, for example between 10 mm and 7 cm. Reaction tubes are understood here to mean vessels whose ratio of length to diameter is greater than 5, preferably between 10 and 100 000, more preferably between 20 and 10 000, for example between 30 and 1000. The length of the reaction tube is understood here to mean the length of the reaction tube over which the microwave irradiation proceeds. Baffles and/or other mixing elements can be incorporated into the reaction tube.

E₀₁ cavity resonators particularly suitable for the process according to the invention preferably have a diameter which corresponds to at least half the wavelength of the microwave radiation used. The diameter of the cavity resonator is preferably 1.0 to 10 times, more preferably 1.1 to 5 times and especially 2.1 to 2.6 times half the wavelength of the microwave radiation used. The E₀₁ cavity resonator preferably has a round cross section, which is also referred to as an E₀₁ round hollow conductor. It more preferably has a cylindrical shape and especially a circular cylindrical shape.

The reaction tube is typically provided at the inlet with a metering pump and a manometer, and at the outlet with a pressure-retaining device and a heat exchanger. This makes possible reactions within a very wide pressure and temperature range.

The preparation of the reaction mixture from carboxylic acid (I), the organic acid (II) bearing at least one amino acid or salt thereof and optionally catalyst and/or solvent can be performed continuously, batchwise or else in semibatchwise processes. Thus, the preparation of the reaction mixture can be performed in an upstream (semi)batchwise process, for example in a stirred vessel. In a preferred embodiment, the reactants, carboxylic acid (I) and organic acid (II) bearing an amine group or salt thereof, and optionally the catalyst, each independently optionally diluted with solvent, are only mixed shortly before entry into the reaction tube. The catalyst can be added to the reaction mixture as such or as a mixture with one of the reactants. For instance, it has been found to be particularly useful to undertake the mixing of carboxylic acid, organic acid bearing an amino group and catalyst in a mixing zone, from which the reaction mixture is conveyed into the reaction tube. Additionally preferably, the reactants and catalyst are supplied to the process according to the invention in liquid form. For this purpose, it is possible to use relatively high-melting and/or relatively high-viscosity reactants, for example in the molten state and/or admixed with solvent, for example in the form of a solution, dispersion or emulsion. The catalyst is added to one of the reactants or else to the reactant mixture before entry into the reaction tube. It is also possible to convert heterogeneous systems by the process according to the invention, in which case appropriate industrial apparatus for conveying the reaction mixture is required.

The reaction mixture can be fed into the reaction tube either at the end conducted through the inner conductor tube or at the opposite end. The reaction mixture can consequently be conducted in a parallel or antiparallel manner to the direction of propagation of the microwaves through the microwave applicator.

By variation of tube cross section, length of the irradiation zone (this is understood to mean the length of the reaction tube in which the reaction mixture is exposed to microwave radiation), flow rate, geometry of the cavity resonator and the microwave power injected, the reaction conditions are preferably established such that the maximum reaction temperature is attained as rapidly as possible and the residence time at maximum temperature remains sufficiently short that as low as possible a level of side reactions or further reactions occurs. To complete the reaction, the reaction mixture can pass through the reaction tube more than once, optionally after intermediate cooling. In the case of slower reactions, it has often been found to be useful to keep the reaction product at reaction temperature for a certain time after it leaves the reaction tube. In many cases, it has been found to be useful when the reaction product is cooled immediately after leaving the reaction tube, for example by jacket cooling or decompression. It has also been found to be useful to deactivate the catalyst immediately after it leaves the reaction tube. This can be accomplished, for example, by neutralization or, in the case of heterogeneously catalyzed reactions, by filtration.

The temperature rise caused by the microwave irradiation is preferably limited to a maximum of 500° C., for example, by regulating the microwave intensity or the flow rate and/or by cooling the reaction tube, for example by means of a nitrogen stream. It has been found to be particularly useful to perform the reaction at temperatures between 150° C. and a maximum of 400° C. and especially between 170° C. and a maximum of 300° C., for example at temperatures between 180° C. and 270° C.

The duration of the microwave irradiation depends on various factors, for example the geometry of the reaction tube, the microwave energy injected, the specific reaction and the desired degree of conversion. Typically, the microwave irradiation is undertaken over a period of less than 30 minutes, preferably between 0.01 second and 15 minutes, more preferably between 0.1 second and 10 minutes and especially between 1 second and 5 minutes, for example between 5 seconds and 2 minutes. The intensity (power) of the microwave radiation is adjusted such that the reaction mixture has the desired maximum temperature when it leaves the cavity resonator. In a preferred embodiment, the reaction product, directly after the microwave irradiation has ended, is cooled as rapidly as possible to temperatures below 120° C., preferably below 100° C. and especially below 60° C.

The reaction is preferably performed at pressures between 1 bar (atmospheric pressure) and 500 bar, more preferably between 1.5 bar and 200 bar, particularly between 3 bar and 150 bar and especially between 10 bar and 100 bar, for example between 15 and 50 bar. It has been found to be particularly useful to work under elevated pressure, which involves working above the boiling point (at standard pressure) of the reactants, products, any solvent present, and/or above the water of reaction formed during the reaction. The pressure is more preferably adjusted to a sufficiently high level that the reaction mixture remains in the liquid state during the microwave irradiation and does not boil.

To avoid side reactions and to prepare products of maximum purity, it has been found to be useful to handle reactants and products in the presence of an inert protective gas, for example nitrogen, argon or helium.

Even though the reactants, carboxylic acid (I) and acid (II) bearing an amino group, often lead to readily manageable reaction mixtures, it has been found to be useful in many cases to work in the presence of solvents in order, for example, to lower the viscosity of the reaction medium and/or to fluidize the reaction mixture, especially if it is heterogeneous. For this purpose, it is possible in principle to use all solvents which are inert under the reaction conditions employed and do not react with the reactants or the products formed. An important factor in the selection of suitable solvents is the polarity thereof, which firstly determines the dissolution properties and secondly the degree of interaction with microwave radiation. A particularly important factor in the selection of suitable solvents is the dielectric loss ε″ thereof. The dielectric loss ε″ describes the proportion of microwave radiation which is converted to heat in the interaction of a substance with microwave radiation. The latter value has been found to be a particularly important criterion for the suitability of a solvent for the performance of the process according to the invention.

It has been found to be particularly useful to work in solvents which exhibit minimum microwave absorption and hence make only a small contribution to the heating of the reaction system. Solvents preferred for the process according to the invention have a dielectric loss ε″ measured at room temperature and 2450 MHz of less than 10 and preferably less than 1, for example less than 0.5. An overview of the dielectric loss of different solvents can be found, for example, in “Microwave Synthesis” by B. L. Hayes, CEM Publishing 2002. Suitable solvents for the process according to the invention are especially those with ε″ values less than 10, such as N-methylpyrrolidone, N,N-dimethylformamide or acetone, and especially solvents with ε″ values less than 1. Examples of particularly preferred solvents with ε″ values less than 1 are aromatic and/or aliphatic hydrocarbons, for example toluene, xylene, ethylbenzene, tetralin, hexane, cyclohexane, decane, pentadecane, decalin, and also commercial hydrocarbon mixtures, such as benzine fractions, kerosene, Solvent Naphtha, Shellsol® AB, Solvesso® 150, Solvesso® 200, Exxsol®, Isopar® and Shellsol® products. Solvent mixtures which have ε″ values preferably below 10 and especially below 1 are equally preferred for the performance of the process according to the invention.

In a further preferred embodiment, the process according to the invention is performed in solvents with higher ε″ values of, for example, 5 or higher, such as especially with ε″ values of 10 or higher, which additionally often exhibit superior dissolution characteristics for the acids (II) bearing amino groups. This embodiment has been found to be useful especially in the conversion of reaction mixtures which themselves, i.e. without the presence of solvents and/or diluents, exhibit only very low microwave absorption. For instance, this embodiment has been found to be useful especially in the case of reaction mixtures which have a dielectric loss ε″ of less than 10 and preferably less than 1. Mixtures of solvents with different ε″ values have also been found to be highly suitable for the inventive reactions. Particularly preferred solvents are lower alcohols having 1 to 5 carbon atoms, for example methanol, ethanol, n-propanol, isopropanol, n-butanol, isobutanol, tert-butanol, the different isomers of pentanol, ethylene glycol, glycerol and water. However, the accelerated heating of the reaction mixture often observed as a result of the solvent addition entails measures to comply with the maximum temperature.

When working in the presence of solvents, the proportion thereof in the reaction mixture is preferably between 2 and 95% by weight, especially between 5 and 90% by weight and particularly between 10 and 75% by weight, for example between 30 and 60% by weight. Particular preference is given to performing the reaction in the presence of polar solvents such as lower alcohols having 1 to 5 carbon atoms or else water.

In a further preferred embodiment, substances which have strong microwave absorption and are insoluble in the reaction mixture are added thereto. These lead to significant local heating of the reaction mixture and, as a result, to further-accelerated reactions. One example of a suitable heat collector of this kind is graphite.

Microwaves refer to electromagnetic rays with a wavelength between about 1 cm and 1 m, and frequencies between about 300 MHz and 30 GHz. This frequency range is suitable in principle for the process according to the invention. For the process according to the invention, preference is given to using microwave radiation with the frequencies approved for industrial, scientific, medical, domestic or similar applications, for example with frequencies of 915 MHz, 2.45 GHz, 5.8 GHz or 24.12 GHz.

The microwave power to be injected into the cavity resonator for the performance of the process according to the invention is especially dependent on the target reaction temperature, but also on the geometry of the reaction tube and hence of the reaction volume, and on the flow rate of the reaction mixture through the reaction tube. It is typically between 200 W and several hundred kW and especially between 500 W and 100 kW, for example between 1 kW and 70 kW. It can be generated by means of one or more microwave generators.

In a preferred embodiment, the reaction is performed in a pressure-resistant, chemically inert tube, in which case the water of reaction which forms and possibly reactants and, if present, solvent lead to a pressure buildup. After the reaction has ended, the elevated pressure can be used, by decompression, for volatilization and removal of water of reaction, excess reactants and any solvent and/or to cool the reaction product. In a further embodiment, the water of reaction formed, after cooling and/or decompression, is removed by customary processes, for example phase separation, filtration, distillation, stripping, flashing and/or absorption.

To achieve particularly high conversions, it has in many cases been found to be useful to expose the reaction product obtained, after removal of water of reaction and optionally removal of product and/or by-product, again to microwave irradiation, in which case the ratio of the reactants used may have to be supplemented to replace consumed or deficient reactants.

The advantages of the process according to the invention lie in very homogeneous irradiation of the reaction mixture in the center of a symmetric microwave field within a reaction tube, the longitudinal axis of which is in the direction of propagation of the microwaves of a monomode microwave applicator and especially within an E₀₁ cavity resonator, for example with a coaxial transition. The inventive reactor design allows the performance of reactions also at very high pressures and/or temperatures. By increasing the temperature and/or pressure, a significant rise in the degree of conversion and yield is observed even compared to known microwave reactors, without this resulting in undesired side reactions and/or discoloration. Surprisingly, a very high efficiency is achieved in the exploitation of the microwave energy injected into the cavity resonator, which is typically above 50%, often above 80%, in some cases above 90% and in special cases above 95%, for example above 98%, of the microwave power injected, and thus gives economic and environmental advantages over conventional preparation processes, and also over prior art microwave processes.

The process according to the invention additionally allows a controlled, reliable and reproducible reaction regime. Since the reaction mixture in the reaction tube is moved parallel to the direction of propagation of the microwaves, known overheating phenomena resulting from uncontrollable field distributions, which lead to local overheating as a result of changing intensities of the microwave field, for example in wave crests and node points, are balanced out by the flowing motion of the reaction mixture. The advantages mentioned also allow working with high microwave powers of more than 1 kW, for example, 2 to 10 kW and especially 5 to 100 kW and in some cases even higher, and hence, in combination with only a short residence time in the cavity resonator, accomplishment of large production volumes of 100 or more tonnes per year in one plant.

It was surprising that, in spite of the only very short residence time of the reaction mixture in the microwave field in the flow tube with continuous flow, very substantial N-acylation with conversions generally of more than 80%, often even more than 90%, for example more than 95%, based on the component used in deficiency, takes place without formation of significant amounts of by-products. It was additionally surprising that the conversions mentioned can be achieved under these reaction conditions without removal of the water of reaction formed in the amidation, and also in the presence of polar solvents such as water and/or alcohols. In case of a corresponding conversion of these reaction mixtures in a flow tube of the same dimensions with thermal jacket heating, extremely high wall temperatures are required to achieve suitable reaction temperatures, which lead to the formation of undefined polymers and colored species, but bring about much lower N-acylation within the same time interval. In addition, the products prepared by the process according to the invention have very low metal contents without any requirement for a further workup of the crude products. For instance, the metal contents of the products prepared by the process according to the invention, based on iron as the main element, are typically below 25 ppm, preferably below 15 ppm, especially below 10 ppm, for example between 0.01 and 5 ppm, of iron.

The process according to the invention thus allows very rapid, energy-saving and inexpensive preparation of amides organic acids which bear amino groups in high yields and with high purity in industrial scale amounts. In this process no significant amounts of by-products are obtained. No unwanted side reactions are observed, for example oxidation of the amine or decarboxylation of the carboxylic acid, which would lower the yield of target product. Such rapid and selective conversions are unachievable by conventional methods and were not to be expected solely through heating to high temperatures.

### EXAMPLES

The conversions of the reaction mixtures under microwave irradiation were effected in a ceramic tube (60×1 cm) which was present in axial symmetry in a cylindrical cavity resonator (60×10 cm). On one of the end sides of the cavity resonator, the ceramic tube passed through the cavity of an inner conductor tube which functions as a coupling antenna. The microwave field with a frequency of 2.45 GHz, generated by a magnetron, was injected into the cavity resonator by means of the coupling antenna (E₀₁ cavity applicator; monomode), in which a standing wave formed.

The microwave power was in each case adjusted over the experiment time in such a way that the desired temperature of the reaction mixture at the end of the irradiation zone was kept constant. The microwave powers mentioned in the experiment descriptions therefore represent the mean value of the microwave power injected over time. The measurement of the temperature of the reaction mixture was undertaken directly after it had left the reaction zone (distance about 15 cm in an insulated stainless steel capillary, Ø1 cm) by means of a Pt100 temperature sensor. Microwave energy not absorbed directly by the reaction mixture was reflected at the opposite end of the cavity resonator from the coupling antenna; the microwave energy which was also not absorbed by the reaction mixture on the return path and reflected back in the direction of the magnetron was passed with the aid of a prism system (circulator) into a water-containing vessel. The difference between energy injected and heating of this water load was used to calculate the microwave energy introduced into the reaction mixture. By means of a high-pressure pump and of a suitable pressure-release valve, the reaction mixture in the reaction tube was placed under such a working pressure which was sufficient always to keep all reactants and products or condensation products in the liquid state. The reaction mixtures prepared from carboxylic acid and alcohol were pumped with a constant flow rate through the reaction tube, and the residence time in the irradiation zone was adjusted by modifying the flow rate.

The products were analyzed by means of ¹H NMR spectroscopy at 500 MHz in CDCl₃. Iron contents were determined by means of atomic absorption spectroscopy.

### Example 1

### Preparation of N-lauroyl-N-methyltaurate

In a 10 l Büchi stirred autoclave with stirrer, internal thermometer and pressure equalizer, 1.6 kg of methyltaurine (10 mol) were dissolved in 4 liters of a water/isopropanol mixture (3:2 parts by volume), and 2.0 kg of lauric acid (10 mol) were added.

The mixture thus obtained was pumped through the reaction tube continuously at 5 l/h at a working pressure of 35 bar and exposed to a microwave power of 2.2 kW, 94% of which was absorbed by the reaction mixture. The residence time of the reaction mixture in the irradiation zone was approx. 34 seconds. At the end of the reaction tube, the reaction mixture had a temperature of 255° C.

A conversion of 83% of theory was attained. The reaction product contained <5 ppm of iron. After distillative removal of isopropanol, a colorless, clear liquid with a high tendency to foam formation was obtained.

### Example 2

### Preparation of N-acetylglycine Sodium Salt

In a 10 l Büchi stirred autoclave with stirrer, internal thermometer and pressure equalizer, 1.0 kg of sodium glycinate (27 mol) dissolved in 2 liters of water were admixed with 3.2 kg of acetic acid (107 mol).

The mixture thus obtained was pumped through the reaction tube continuously at 5 l/h at a working pressure of 30 bar and exposed to a microwave power of 1.8 kW, 92% of which was absorbed by the reaction mixture. The residence time of the reaction mixture in the irradiation zone was approx. 34 seconds. At the end of the reaction tube, the reaction mixture had a temperature of 261° C.

A conversion of 90% of theory was attained. The reaction product contained <5 ppm of iron.

### Example 3

### Preparation of N-stearoylglycine Sodium Salt

In a 10 l Büchi stirred autoclave with stirrer, internal thermometer and pressure equalizer, 1.2 kg of glycine sodium salt (12 mol) were dissolved in 3.5 liters of a water/isopropanol mixture (2:2 parts by volume) and admixed with 2.65 kg of stearic acid (9.3 mol).

The mixture thus obtained was pumped through the reaction tube continuously at 4 l/h at a working pressure of 35 bar and exposed to a microwave power of 2.6 kW, 90% of which was absorbed by the reaction mixture. The residence time of the reaction mixture in the irradiation zone was approx. 42 seconds. At the end of the reaction tube, the reaction mixture had a temperature of 267° C.

A conversion of 79% of theory was attained. The reaction product contained <5 ppm of iron.

### Example 4

### Preparation of 4-(N-cocoyl)amidobenzoic acid

In a 10 l Büchi stirred autoclave with stirrer, internal thermometer and pressure equalizer, 1.45 kg of 4-aminobenzoic acid (10.5 mol) and 2.25 kg of coconut fatty acid (10.5 mol) were dissolved in 5 l of isopropanol while heating.

The mixture thus obtained was pumped through the reaction tube continuously at 3.5 l/h at a working pressure of 35 bar and exposed to a microwave power of 1.6 kW, 87% of which was absorbed by the reaction mixture. The residence time of the reaction mixture in the irradiation zone was approx. 49 seconds. At the end of the reaction tube, the reaction mixture had a temperature of 281° C.

A conversion of 85% of theory was attained. The reaction product contained <5 ppm of iron.

1. A continuous process for N-acylation of organic acids bearing amino groups, in which at least one carboxylic acid of the formula (I)
R¹—COOH (I)
in which
R¹ is hydrogen or an optionally substituted hydrocarbyl radical having 1 to 50 carbon atoms,
is reacted with at least one organic acid which bears at least one amino group and is of the formula (II)
R²NH-A-X (II)
in which
A is an optionally substituted hydrocarbyl radical having 1 to 50 carbon atoms
X is an acid group or the metal salt thereof, and
R² is hydrogen, an optionally substituted hydrocarbyl radical having 1 to 50 carbon atoms or a group of the formula -A-X in which A and also X are each independently as defined above,
under microwave irradiation in a reaction tube whose longitudinal axis is in the direction of propagation of the microwaves from a monomode microwave applicator to give the amide.

2. The process as claimed in claim 1, in which the reaction mixture is irradiated with microwaves in a substantially microwave-transparent reaction tube within a hollow conductor connected via waveguides to a microwave generator.

3. The process as claimed in one or more of claims 1 and 2, in which the microwave applicator is configured as a cavity resonator.

4. The process as claimed in one or more of claims 1 to 3, in which the microwave applicator is configured as a cavity resonator of the reflection type.

5. The process as claimed in one or more of claims 1 to 4, in which the reaction tube is aligned axially with a central axis of symmetry of the hollow conductor.

6. The process as claimed in one or more of claims 1 to 5, in which the reaction mixture is irradiated in a cavity resonator with a coaxial transition of the microwaves.

7. The process as claimed in one or more of claims 1 to 6, in which the cavity resonator is operated in E₀₁ₙ mode where n is an integer from 1 to 200.

8. The process as claimed in one or more of claims 1 to 7, in which a standing wave forms in the cavity resonator.

9. The process as claimed in one or more of claims 1 to 8, in which the reaction mixture is heated by the microwave irradiation to temperatures between 150 and 500° C.

10. The process as claimed in one or more of claims 1 to 9, in which the microwave irradiation is effected at pressures above atmospheric pressure.

11. The process as claimed in one or more of claims 1 to 10, in which R¹ is an optionally substituted aliphatic hydrocarbyl radical having 2 to 30 carbon atoms.

12. The process as claimed in one or more of claims 1 to 11, in which R¹ is an optionally substituted aliphatic hydrocarbyl radical which has 2 to 30 carbon atoms and contains at least one C═C double bond.

13. The process as claimed in one or more of claims 1 to 11, in which R¹ is a saturated alkyl radical having 1, 2, 3 or 4 carbon atoms.

14. The process as claimed in one or more of claims 1 to 12, in which R¹ is an optionally substituted alkenyl group having 2 to 4 carbon atoms.

15. The process as claimed in one or more of claims 1 to 10, in which R¹ is an optionally substituted cyclic through-conjugated system having (4n+2) π electrons where n is 1, 2, 3, 4 or 5.

16. The process as claimed in one or more of claims 1 to 10, in which the carboxylic acid of the formula I is selected from formic acid, acetic acid, propionic acid, butyric acid, isobutyric acid, pentanoic acid, isopentanoic acid, pivalic acid, acrylic acid, methacrylic acid, crotonic acid, 2,2-dimethylacrylic acid, maleic acid, fumaric acid, itaconic acid, cinnamic acid, methoxycinnamic acid, succinic acid, butanetetracarboxylic acid, phenylacetic acid, (2-bromophenyl)acetic acid, (methoxyphenyl)acetic acid, (dimethoxyphenyl)acetic acid, 2-phenylpropionic acid, 3-phenylpropionic acid, 3-(4-hydroxyphenyl)propionic acid, 4-hydroxyphenoxyacetic acid, hexanoic acid, cyclohexanoic acid, heptanoic acid, octanoic acid, nonanoic acid, neononanoic acid, decanoic acid, neodecanoic acid, undecanoic acid, neoundecanoic acid, dodecanoic acid, tridecanoic acid, tetradecanoic acid, 12-methyltridecanoic acid, pentadecanoic acid, 13-methyltetradecanoic acid, 12-methyltetradecanoic acid, hexadecanoic acid, 14-methylpentadecanoic acid, heptadecanoic acid, 15-methylhexadecanoic acid, 14-methylhexadecanoic acid, octadecanoic acid, isooctadecanoic acid, eicosanoic acid, docosanoic acid, tetracosanoic acid, myristoleic acid, palmitoleic acid, hexadecadienoic acid, delta-9-cis-heptadecenoic acid, oleic acid, petroselic acid, vaccenic acid, linoleic acid, linolenic acid, gadoleic acid, gondoic acid, eicosadienoic acid, arachidonic acid, cetoleic acid, erucic acid, docosadienic acid and tetracosenoic acid, dodecenylsuccenic acid, octadecenylsuccenic acid, carboxylic acid mixtures obtained from cottonseed oil, coconut oil, peanut oil, safflower oil, corn oil, palm kernel oil, rapeseed oil, olive oil, mustardseed oil, soybean oil, sunflower oil, tallow oil, bone oil and fish oil, tall oil fatty acid, resin acids and naphthenic acids, benzoic acid, phthalic acid, isophthalic acid, the isomers of naphthalenecarboxylic acid, pyridinecarboxylic acid and naphthalenedicarboxylic acid, trimellitic acid, trimesic acid, pyromellitic acid and mellitic acid, the isomers of methoxybenzoic acid, hydroxybenzoic acid, hydroxymethylbenzoic acid, hydroxymethoxybenzoic acid, hydroxydimethoxybenzoic acid, hydroxyisophthalic acid, hydroxynaphthalenecarboxylic acid, hydoxypyridinecarboxylic acid, hydroxymethylpyridinecarboxylic acid, hydroxyquinolinecarboxylic acid, o-toluic acid, m-toluic acid, p-toluic acid, o-ethylbenzoic acid, m-ethylbenzoic acid, p-ethylbenzoic acid, o-propylbenzoic acid, m-propylbenzoic acid, p-propylbenzoic acid and 3,4-dimethylbenzoic acid.

17. The process as claimed in one or more of claims 1 to 16, in which A is selected from aliphatic radicals having 1 to 12 carbon atoms and aromatic radicals having 5 to 12 carbon atoms.

18. The process as claimed in one or more of claims 1 to 17, in which R² is selected from the group consisting of H, optionally substituted aliphatic radicals having 2 to 18 carbon atoms, optionally substituted C₆-C₁₂-aryl groups, optionally substituted heteroaromatic groups having 5 to 12 ring members, or a group of the formula -A-X where
A is an optionally substituted hydrocarbyl radical having 1 to 50 carbon atoms and
X is an acid group or the metal salt thereof.

19. The process as claimed in one or more of claims 1 to 18, in which X is selected from the group consisting of carboxylic acids, sulfonic acids and phosphonic acids.

20. The process as claimed in one or more of claims 1 to 19, in which X is an alkali metal or alkaline earth metal salt of an acid group.

21. The process as claimed in one or more of claims 1 to 20, in which the organic acid which bears at least one amino group and is of the formula (II) is selected from α-aminocarboxylic acids, β-aminosulfonic acids, aminomethylenephosphonic acids and metal salts thereof.

22. The process as claimed in one or more of claims 1 to 21, in which carboxylic acid (I) and organic acid (II) bearing an amino group are reacted in a molar ratio of 20:1 to 1:20, based in each case on the molar equivalents of carboxyl and amino groups.

23. The process as claimed in one or more of claims 1 to 22, which is performed in the presence of basic catalysts.
